(19) United States
(12) Patent Application Publication
(10) Pub. No.: US 2005/0250801 A1
(43) Pub. Date: Nov. 10, 2005

(54) Method of treating cancer with azaspirane compositions

(76) Inventors: Kunwar Shailubhai, Norristown, PA (US); Gary S. Jacob, New York, NY (US); Donald H. Picker, Forrest Hills, NY (US); Geoffrey W. Henson, Ferndale, WA (US); Simon Fricker, Langley (CA)

Correspondence Address: PILLSBURY WINTHROP SHAW PITTMAN, LLP, P.O. BOX 10500, MCLEAN, VA, 22102, US

(21) Appl. No.: 10/796,292

(22) Filed: Mar. 10, 2004

Related U.S. Application Data
(60) Provisional application No. 60/452,951, filed on Mar. 10, 2003.
(60) Provisional application No. 60/474,929, filed on Jun. 3, 2003.

(51) Int. Cl. A61K 31/4747; A61K 31/403

(52) U.S. Cl. 514/278; 514/409

(57) ABSTRACT

A method of treating cancer by administering a therapeutically effective amount of a compound represented by the following Formula (I), or salt, hydrate, or solvate thereof: 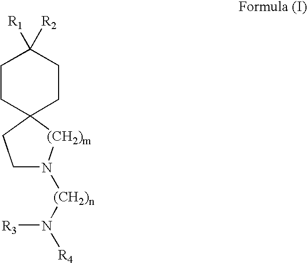 wherein: n represents a number from 3 to 7; m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5 and no greater than 10; or R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical.

### CROSS REFERENCE TO RELATED APPLICATIONS

This application claims the benefit of priority from U.S. Provisional Ser. No. 60/452,951, filed Mar. 10, 2003, and U.S. Provisional Ser. No. 60/474,929, filed Jun. 3, 2003, which are incorporated, in their entirety, herein by reference.

### FIELD OF THE INVENTION

This invention relates to the use of certain azaspiranes as therapeutics for treating cancer. In particular, this invention relates to treating cancer in mammals, including humans, by regulating or controlling, for example, angiogenesis and/or apoptosis by administering certain azaspiranes defined herein.

### BACKGROUND OF THE INVENTION

Cancers are a major cause of death in animals and humans. The exact cause of cancer is not known, but links between certain activities such as smoking or exposure to carcinogens and the incidence of certain types of cancers has been shown by a number of researchers.

Many types of chemotherapeutic agents have been shown to be effective against cancers, but not all types of cancers respond to these agents. Unfortunately, many of these agents also destroy normal cells. The exact mechanisms for the action of these chemotherapeutic agents are not always known.

Despite advances in the field of cancer treatment the leading therapies to date are surgery, radiation and chemotherapy. Chemotherapeutic approaches are said to fight cancers that are metastasized or ones that are particularly aggressive. Such cytocidal or cytostatic agents work best on cancers with large growth factors, i.e., ones whose cells are rapidly dividing. To date, hormones, in particular estrogen, progesterone and testosterone, and some antibiotics produced by a variety of microbes, alkylating agents, and anti-metabolites form the bulk of therapies available to oncologists.

Compelling evidence implicates angiogenesis may play a role in both tumor growth and metastasis, as well as in several other human diseases, such as diabetic retinopathy, rheumatoid arthritis and psoriasis. Angiogenesis is a multiple-step process that an organism uses to form new blood vessels from preexisting vasculature. These steps are activated by angiogenic stimulus by growth factors and cytokines. See: Folkman, J. What is the Evidence that Tumors are Angiogenesis-Dependent? J. Natl. Cancer Inst. 1991, 82, 4-6; Folkman, J. Angiogenesis in Cancer, Vascular, Rheumatoid and Other Disease. Nat. Med 1995, 1, 27-31; McDonnell, C. O.; Hill, A. D. K.; McNamara, D. A.; Walsh, T. N.; Bouchier-Hayes, D. J. Tumor Micrometastases: The Influence of Angiogenesis. Eur. J. Surg. Oncol. 2000, 26, 105-115; Li, W. Tumor Angiogenesis: Molecular Pathology, Therapeutic Targeting, and Imaging. Acad Radiol. 2000, 7, 800-811; Kerbel, R. S. Tumor Angiogenesis: Past, Present and the Near Future. Carcinogenesis 2000, 21, 505-515; Carmeliet, P.; Jam, R. K. Angiogenesis in Cancer and Other Diseases. Nature 2000, 407, 249-257.

Normally, angiogenesis ceases when the initial angiogenic signals subside and other, secondary, signals predominate to turn off the angiogenic process. However in disease states such as cancer, the local concentration of angiogenic signals never decreases and new blood vessels continuously form. Therefore undesired angiogenesis provides a steady supply of nutrients to the tumor, allowing the tumor to grow as well as metastasize. See: Folkman, J. Angiogenesis in cancer, vascular, rheumatoid and other diseases. Nat. Med. 1, 27-31, 1995; Folkman J. Tumor angiogenesis: a possible control point in tumor growth. Ann Intern Med. 1975; 82:96-100; Folkman J, Watson K, Ingber D, Hanahan D. Induction of angiogenesis during the transition from hyperplasia to neoplasia. Nature. 1989; 339:58-61; Folkman J. What is the evidence that tumors are angiogenesis dependent? J Natl Cancer Inst. 1989; 82:4-6; Folkman J. Ingber D E. Angiostatic steroids: method of discovery and mechanism of action. Ann Surg. 1987;206:374-383; Barger A C, Beeuwkes R. Lainey L L. Silverman K). Hypothesis: vasavasorum and neovascularization of human coronary arteries. N Engl. J. Med. 1984:310:175-177; Heistad D H, Armstrong M L. Bloodflow through vasa vasorum of coronary arteries in atherosclerotic monkeys. Arteriosclerosis. 1986: 6:326-331; O'Brien E R, Garvin M R. Dev R, Stewart D K, Hinohara T. Simpson, J B. Shwartz S M. Angiogenesis in human coronary atherosclerotic plaques. Am J Pathol. 1994; 145:883-894; Saaristo A, Karpanen T, Alitalo K. Mechanisms of angiogenesis and their use in the inhibition of tumor growth and metastasis. Oncogene. 19, 6122-6129, 2000.

A number of growth factors have been identified as potential positive regulators of angiogenesis, including vascular endothelial growth factor (VEGF), basic fibroblast growth factor (bFGF), transforming growth factor α (TGFα), TGFβ, tumor necrosis factor, platelet-derived endothelial growth factor, hepatocyte growth factor, angiogenin, interleukin-8 and placenta growth factor. At least two of these angiogenic factors, bFGF and VEGF, are able to induce angiogenesis in vivo. See: Klagsbrun, M.; D'Amore, P. A. Regulators of Angiogenesis. Annu. Rev. Physiol. 1991, 53, 2 17-239; Gerwins, P.; Skoldenberg, E.; Clacsson-Welsh, L. Function of Fibroblast Growth Factors and Vascular Endothelial Growth Factors and Their Receptors in Angiogenesis. Crit. Rev, Oncol. Hematol. 2000, 34, 185-194; Liekens, S.; Clercq, E. D.; Neyts. 1. Angiogenesis Regulators and Clinical Applications. Biochem. Pharmacol. 2091, 61, 253-270; Leung, D. W.; Cachianes. C.; Kuang. W. J.; Goeddel, D. V., Ferrara, N. Vascular Endothelial Growth Factor is A Secreted Angiogenic Mitogen. Science 1989, 246, 1306-1313; Ferrara N. The Role of Vascular Endothelial Growth Factor in Pathological Angiogenesis. Breast Cancer Res. Treat. 1995, 36, 127-137; Ferrara N. Vascular Endothelial Growth Factor. Trends Cardiovasc. Med 1993, 3, 244-250.

Clinically, high circulating levels of bFGF and VEGF have been correlated with promotion and progression of certain tumors. VEGF is distinct among these growth factors in that it acts as an endothelial cell-specific mitogen; and it is the one growth factor most consistently found in a wide variety of conditions associated with angiogenesis. See: Heistad D H, Armstrong M L. Bloodflow through vasa vasorum of coronary arteries in atherosclerotic monkeys. Arteriosclerosis. 1986: 6:326-331; O'Brien E R, Garvin M R. Dev R, Stewart D K, Hinohara T. Simpson, J B. Shwartz S M. Angiogenesis in human coronary atherosclerotic plaques. Am J Pathol. 1994; 145:883-894; Saaristo A, Karpanen T, Alitalo K. Mechanisms of angiogenesis and their use in the inhibition of tumor growth and metastasis. Oncogene. 19, 6122-6129, 2000; Folkman, J. Angiogenesis in cancer, vascular, rheumatoid and other diseases. Nat. Med. 1, 27-31, 1995; Klagsbrun, M.; D'Amore, P. A. Regulators of Angiogenesis. Annu. Rev. Physiol. 1991, 53, 2 17-239; Gerwins, P.; Skoldenberg, E.; Clacsson-Welsh, L. Function of Fibroblast Growth Factors and Vascular Endothelial Growth Factors and Their Receptors in Angiogenesis. Crit. Rev, Oncol. Hematol. 2000, 34, 185-194. In benign colorectal adenomas, VEGF protein and transcript levels exceed those of normal colonic mucosa. See: Lee J C, Chow N H, Wang S T, Huang S M. Prognostic value of vascular endothelial growth factor expression in colorectal cancer patients. Eur. J. Cancer. 2000, 36:748-753.

Inhibition of VEGF activity, or disabling the function of its receptors, has been shown to inhibit both tumor growth and metastasis in a variety of animal tumor models. For example, VEGF levels are significantly higher in metastatic colorectal tumors. These findings suggest that VEGF and its receptors play an important role in tumor angiogenesis, and therefore are excellent targets for human disease intervention where pathological angiogenesis is involved. See: Brown L F, Detmar M, Claffey K, Nagy J A, Feng D, Dvorak A M, Dvorak H F. Vascular permeability factor/vascular endothelial growth factor: a multifunctional angiogenic cytokine. EXS. 79, 233-269, 1997; Cascinu, S., Graziano, F., Catalano, V., Staccioli, M. P., Barni, S., Giordani, P., Rossi, M. C., Baldelli, A. M., Muretto, P., Valenti, A., and Catalano, G. Differences of vascular endothelial growth factor (VEGF) expression between liver and abdominal metastases from colon cancer. Implications for the treatment with VEGF inhibitors. Clin Exp Metastasis. 18, 651-655, 2000.

Research into apoptosis (programmed cellular death) has also provided insight into mechanisms of cancer. For example, disruption of the normal turnover of epithelial cells lining the gastrointestinal mucosa through disregulated apoptosis and irregular proliferation is thought to lead to colon cancer. An example of this is the correlation of higher proliferative index with colorectal cancer. See: Askling, J., Dickman, P. W., Karlen, P., Brostrom, O., Lapidus, A., Lofberg, R., and Ekbom, A. Colorectal cancer rates among first-degree relatives of patients with inflammatory bowel disease: a population-based cohort study. Lancet, 357: 262-266, 2001. More specifically, evidence indicates that stem cells at the base of gastrointestinal crypts proliferate and differentiate as they migrate along the walls of the crypts, ultimately functioning as fully differentiated goblet cells and absorptive epithelial cells. These mature cells are continually turned over to rejuvenate the epithelial layer of the gastrointestinal mucosa by the process of apoptosis, after which they are engulfed by stromal cells or shed into the GI lumen. See: Provenzalen, D. and Onken, J. Surveillance issues in inflammatory bowel disease: Ulcerative colitis. J Clin Gastroenterol, 32:99-105, 2001.

Reduced rates of apoptosis are often associated with abnormal growth, inflammation, and neoplastic transformation. Homeostasis in GI mucosa, for example, is regulated by equal rates of cell proliferation and apoptosis; disruption of this process by increased cell proliferation and/or decreased apoptosis could lead to generation of adenomas and subsequently to adenocarcinomas. See: Eastwood G L. Epithelial renewal in premalignant conditions of the gastrointestinal tract: a review. J Clin Gastroenterol. 14, S29-33, 1992. Hence, therapeutic agents that inhibit proliferation and induce apoptosis are attractive candidates for cancer treatment.

A cell is believed to initiate apoptosis by activating specific cellular proteases (caspases). Hence, activation of caspases may serve as a signal for induction of apoptosis. Therefore, therapeutic agents that activate pro-apoptotic enzymes (e.g. caspases-3 and caspases-9) are considered to be anti-cancer agents. See: Hughes, F. M. Jr., and Cidlowski, J. A. Potassium is a critical regulator of apoptotic enzymes in vitro and in vivo. Adv. Enzyme Regul., 39:157-171, 1999; Bortner, C. D., Hughes, F. M. Jr., and Cidlowski, J. A. A primary role for K⁺ and Na⁺ efflux in the activation of apoptosis. J. Biol. Chem., 272:32436-32442, 1997.

### SUMMARY OF INVENTION

One embodiment of the present invention provides a method of treating cancer comprising administering to a mammal a therapeutically effective amount of a compound represented by the following Formula (I), or pharmaceutically acceptable salt, hydrate, or solvate thereof: 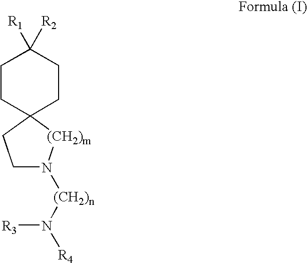
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical or
R₃ and R₄ together with the nitrogen represent at least a 4-member heterocyclic group.

In another embodiment of the present invention, a method of treating cancer is provided by administering a Compound represented by Formula I in combination with a chemotherapeutic or potentiating agent. A further embodiment of the present invention includes the treatment of cancer by administering a Compound having a percent inhibition of proliferation of CaCo-2 cells at 5 μM, of greater than 45%, including, for example, greater than 50%, 60%, 70% or 80%.

In another embodiment of the present invention, a method of inhibiting the proliferation of cancer cells is provided by administering a Compound represented by Formula I. Another embodiment of the present invention provides a method of accelerating the rate of apoptosis in cancer cells is provided by administering a therapeutically acceptable amount of a Compound represented by Formula I. A still further embodiment of the present invention is a method of inhibiting the secretion of VEGF by administering a therapeutically acceptable amount of a Compound represented by Formula I. Another embodiment of the present invention provides a method for inhibiting or even stopping angiogenesis by administering a therapeutically acceptable amount of a Compound represented by Formula 1.

Additional objects, advantages and features of the present invention are set forth in this specification, and in part will become apparent to those skilled in the art on examination of the following, or may be learned by practice of the invention. The inventions disclosed in this application are not limited to any particular set of or combination of objects, advantages and features. It is contemplated that various combinations of the stated objects, advantages and features make up the inventions disclosed in this application.

### BRIEF DESCRIPTION OF DRAWINGS

FIG. 1 is a graph showing the inhibition of proliferation of (a) CaCo-2 and (b) T84 cells by N,N,-diethyl-8,8-dipropyl-2-azaspiro[4,5]decane-2-propanamine dimaleate (Compound 1).

FIG. 2: is a graph showing the inhibition of proliferation of HUVEC cells by N,N,-diethyl-8,8-dipropyl-2-azaspiro[4,5]decane-2-propanamine dimaleate (Compound 1).

FIG. 3: is a DNA fragmentation micrograph showing induction of apoptosis in T84 and CaCo-2 cells by N,N,-diethyl-8,8-dipropyl-2-azaspiro[4,5]decane-2-propanamine dimaleate (Compound 1).

FIG. 4: is a DNA fragementation micrograph showing induction of apoptosis in HUVEC cells by N,N,-diethyl-8,8-dipropyl-2-azaspiro[4,5]decane-2-propanamine dimaleate (Compound 1).

FIG. 5: is a graph showing activation of caspase-3 and caspase-9 by N,N,-diethyl-8,8-dipropyl-2-azaspiro[4,5]decane-2-propanamine dimaleate (Compound 1).

FIG. 6: is a compilation of graphs showing tumor cell growth as a function of N,N,-diethyl-8,8-dipropyl-2-azaspiro[4,5]decane-2-propanamine dimaleate (Compound 1) concentration.

FIG. 7: is a graph showing the mean excretion of radioactivity following single oral administration of [¹⁴C] N,N,-diethyl-8,8-dipropyl-2-azaspiro[4,5]decane-2-propanamine dimaleate salt, (“Compound II”) to male rats at a target dose level of 1 mg free base/kg.

FIG. 8: is a graph showing HUVEC cell proliferation as a function of N,N,-diethyl-8,8-dipropyl-2-azaspiro[4,5]decane-2-propanamine dimaleate (Compound 1) concentration, relative to a control.

FIG. 9: is a graph showing HUVEC cord formation as a function of N,N,-diethyl-8,8-dipropyl-2-azaspiro[4,5]decane-2-propanamine dimaleate (Compound 1) concentration, relative to a control.

FIG. 10: is a graph showing HUVEC cell migration as a function of N,N,-diethyl-8,8-dipropyl-2-azaspiro[4,5]decane-2-propanamine dimaleate (Compound 1) concentration, relative to a control.

### DETAILED DESCRIPTION OF THE INVENTION

As used herein the following terms, unless otherwise specified, are understood to have the following meanings:

“Compound” refers to the compound or salt, hydrate, or solvate thereof. For example, the usage of the term Compound as in “a Compound represented by Formula 1” will be understood to mean “a compound represented by Formula 1 or pharmaceutically acceptable salt, hydrate, or solvate thereof”.

“HUVEC” refers to a Human Umbilical Vein Endothelial Cell(s).

“parenteral” as used herein includes intravenous, intramuscular, subcutaneous, intranasal, intrarectal, intravaginal or intraperitoneal administration.

“pharmaceutically acceptable” refers to substances that, when taking into account the benefits versus the risks, are acceptable for use with mammals, including humans, without undue adverse side effects (such as toxicity, irritation, and allergic response).

“cancer” refers to an abnormal growth of cells which tend to proliferate in an uncontrolled way, including neoplasms, tumors and leukemia. Preferably, the methods of the present invention include treatment of leukemias, melanomas, carcinomas and sarcomas. Additional exemplary cancers include cancer of the brain, breast, pancreas, cervix, colon, head & neck, kidney, lung, non-small cell lung, melanoma, mesothelioma, ovary, sarcoma, stomach, uterus, liver, testicles, mouth, and medulloblastoma.

“leukemia” refers broadly to diseases of the blood-forming organs and is generally characterized by a distorted proliferation and development of leukocytes and their precursors in the tissues, blood and/or bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present invention includes a method of treating leukemia by administering a therapeutically acceptable amount of a Compound represented by Formula 1. For example, the present invention embodies methods of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

“sarcoma” generally refers to a cancerous growth comprising an embryonic-connective-tissue like substance and is generally composed of closely packed cells embedded in a fibrillar or homogeneous substance. Sarcomas can be treated by the administration of a therapeutically acceptable amount of a Compound represented by Formula 1. Specific Sarcomas that may be treated by this method include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“melanoma” generally refers to a cancerous growth arising from the melanocytic system of the skin and other organs. Melanoma can be treated by the administration of a therapeutically acceptable amount of a Compound represented by Formula 1. Specific Melanoma that may be treated by this method include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

“carcinoma” generally refers to a cancerous growth made up of epithelial cells tending to infiltrate the surrounding tissues and give rise to metastasis. Carcinoma can be treated by the administration of a therapeutically acceptable amount of a Compound represented by Formula 1. Specific Carcinomas that may be treated by this method include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers which can be treated with the administration of a therapeutically acceptable amount of a Compound represented by Formula 1 include, but are not limited to, Hodgkin's Disease, Non-Hodgkin's Lymphoma, adenocarcinoma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, glioblastoma, esophageal cancer, genitourinary tract cancer, malignant hypercalcemia, cervical cancer, endometrial cancer, adrenal cortical cancer, and prostate cancer.

The compounds useful in the methods of the present invention comprise compounds represented by the following Formula (I), or a salt, hydrate, or solvate thereof: 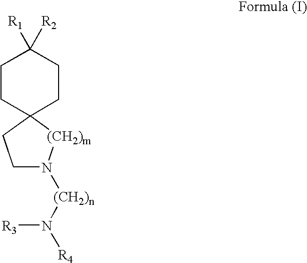
wherein:
n represents a number from 3 to 7, for example 3, 4 or 6;
m represents a number from 1 to 2, for example 1;
R₁ and R₂ independently represent a hydrogen atom or are a substituted or unsubstituted, branched or unbranched or cyclic, alkyl provided that the total number of carbon atoms represented by R₁ and R₂ when taken together is no less than 5 or between 5 and 12, for example 6, 8 or 10; or R₁ and R₂ together independently represent a cyclic alkyl group having no less than 3 or no more than 7 carbon atoms; for example wherein R₁ and R₂ independently represent an unsubstituted alkyl, an unbranched alkyl, a branched or unbranched or cyclic 1 to 5 carbon alkyl, ethyl, propyl, butyl, pentyl or hexyl; and
R₃ and R₄ independently represent a hydrogen atom or a saturated or unsaturated, substituted or unsubstituted, branched or unbranched or cyclic, hydrocarbyl radical; for example, wherein at least one of said R₃ or R₄ independently includes an alkyl or a hydrogen atom or a straight chain alkyl having no less than 1 and no more than 3 carbon atoms, methyl, ethyl, propyl, or R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group, for example a 5 to 8-member heterocyclic group including a 6-member heterocyclic group.

The preparation of compounds represented by Formula (1) and pharmaceutically acceptable salts, hydrates and solvates thereof is disclosed in U.S. Pat. Nos. 4,963,557; 5,734,061; 5,744,495; 5,939,450 and 5,952,365 the entire disclosures of which are incorporated herein by reference.

Typically, a Compound represent by Formula (I) is administered in admixture with suitable pharmaceutical diluents, extenders, excipients, or carriers (collectively referred to herein as a pharmaceutically acceptable carriers or carrier materials) suitably selected with respect to the intended form of administration and as consistent with conventional pharmaceutical practices. The unit will usually be in a form suitable for oral, rectal, topical, intravenous injection or parenteral administration.

A compound represented by Formula (I) may be administered alone but is generally mixed with a pharmaceutically acceptable carrier. This carrier can be a solid or liquid, and the type of carrier is generally chosen based on the type of administration being used. Specific examples of pharmaceutical acceptable carriers and excipients that may be used to formulate oral dosage forms of the present invention are described in U.S. Pat. No. 3,903,297 to Robert, issued Sep. 2, 1975, which is hereby incorporated herein, in its entirety, by reference. Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.) all of which are hereby incorporated herein by reference.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like.

Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

In addition to the Compound, such compositions may contain pharmaceutically acceptable carriers and other ingredients known to facilitate administration and/or enhance uptake. Other formulations, such as microspheres, nanoparticles, liposomes, and immunologically-based systems may also be used in accordance with the present invention. Other examples include formulations with polymers (e.g., 20% w/v polyethylene glycol) or cellulose, or enteric formulations.

Additional pharmaceutically acceptable carries and examples of pharmaceutically acceptable tablets, capsules, suspensions and kits may be found in U.S. Pat. No. 6,384,049, which is hereby incorporated herein in its entirety by reference.

In some embodiments, the Compounds represented by Formula (I) are used in combination with one or more potentiators and/or chemotherapeutic agents. These combinations can be administered together or sequentially. An exemplary potentiator, for use in the present invention, includes triprolidine or its cis-isomer. Triprolidine is described in U.S. Pat. No. 5,114,951 (1992) which is hereby incorporated, in its entirety, by reference. Other suitable potentiators, for use in the present invention, include procodazole, 1H-benzimidazole carbamate-2-propanoic acid; [β-(2-benzimidazole carbamate) propionic acid; 2-(2-carboxyethyl)benzimidazole carbamate; propazol]. Procodazole is a non-specific immunoprotective agent active against viral and bacterial infections.

Other potentiators which can be used with the Compounds represented by Formula (I), and optionally another chemotherapeutic agent, in the treatment methods of the present invention include monensin, an anti-sense inhibitor of the RAD51 gene, bromodeoxyuridine, dipyridamolle indomethacin, a monoclonal antibody, an anti-transferrin receptor immunotoxin, metoclopramide, 7-thia-8-oxoguanosine, N-solanesyl-N,N′-bis(3,4-dimethoxybenzyl)ethylenediamine, leucovorin, heparin, N-[4-[(4-fluorphenyl)sulfonly]phenyl]acetamide, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative, and dimethyl sulfoxide.

Suitable chemotherapeutic agents which can be used with the Compounds of Formula (I), and optionally potentiators, are generally grouped as DNA-interactive agents, antimetabolites, tubulin-interactive agents, hormonal agents and others such as asparaginase or hydroxyurea. For a detailed discussion of chemotherapeutic agents and their method of administration that can be used with the presented invention, see Dorr, et al, Cancer Chemotherapy Handbook, 2d edition, pages 15-34, Appleton & Lange (Connecticut, 1994) which is hereby incorporated by reference.

Suitable DNA-interactive agents include the alkylating agents, e.g., Cisplatin, Cyclophosphamide, Altretamine; the DNA strand-breakage agents, such as Bleomycin; the intercalating topoisomerase II inhibitors, (e.g., Dactinomycin and Doxorubicin); the nonintercalating topoisomerase II inhibitors such as, Etoposide and Teniposde; and the DNA minor groove binder Plicamydin.

Alkylating agents form covalent chemical adducts with cellular DNA, RNA, and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. Suitable alkylating agents include: nitrogen mustards, such as Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin or Carboplatin; bioreductive alkylator, such as Mitomycin, and Procarbazine; Dacarbazine and Altretamine.

Suitable DNA strand breaking agents include Bleomycin.

Suitable DNA topoisomerase II inhibitors include the following: intercalators, such as Amsacrine, Dactinomycin, Daunorubicin, Doxorubicin, Idarubicin, and Mitoxantrone; and nonintercalators, such as Etoposide and Teniposide. Suitable DNA minor groove binder includes Plicamycin.

Antimetabolites interfere with the production of nucleic acids by one or the other of two major mechanisms. Some of the drugs inhibit production of the deoxyribonucleoside triphosphates that are the immediate precursors for DNA synthesis, thus inhibiting DNA replication. Some of the compounds are sufficiently like purines or pyrimidines to be able to substitute for them in the anabolic nucleotide pathways. These analogs can then be substituted into DNA and RNA instead of their normal counterparts. The antimetabolites useful herein include: folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea.

Tubulin interactive agents act by binding to specific sites on tubulin, a protein that polymerizes to form cellular microtubules. Microtubules are critical cell structure units. When the interactive agents bind on the protein, the cell can not form microtubules. Suitable tubulin interactive agents include colchicine, Vincristine and Vinblastine, both alkaloids and Paclitaxel and cytoxan.

Hormonal agents are also useful in the treatment of cancers and tumors. They are used in hormonally susceptible tumors and are usually derived from natural sources. Suitable hormonal agents for use in the methods of the present invention include: estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, methyltestosterone.

Adrenal corticosteroids are derived from natural adrenal cortisol or hydrocortisone. They are used because of their anti-inflammatory benefits as well as the ability of some to inhibit mitotic divisions and to halt DNA synthesis. Suitable adrenal cortocosteriods useful in the methods of the present invention include prednisone, dexamethasone, methylprednisolone, and prednisolone.

Leutinizing hormone releasing agents or gonadotropin-releasing hormone antagonists are used primarily for the treatment of prostate cancer. Suitable components for use in the methods of the present invention include leuprolide acetate and goserelin acetate.

Suitable antihormonal antigens include: antiestrogenic agents such as Tamoxifen, antiandrogen agents such as Flutamide; and antiadrenal agents such as Mitotane and Aminoglutethimide.

Hydroxyurea, which appears to act primarily through inhibition of the enzyme ribonucleotide reductase, can also be used in combination with the methods of the present invention.

Asparaginase is an enzyme which converts asparagine to nonfunctional aspartic acid and thus blocks protein synthesis in the tumor. Asparaginase can also be used in combination with the Compounds of Formula (I) in the methods of the present invention.

A compound represented by Formula (I) or a pharmaceutically acceptable salt or hydrate or solvate thereof is administered to a mammal, including a human, to treat cancers of that mammal. The administration method may include, for example, oral or parenteral.

It will be recognized by one of skill in the art that the optimal quantity and spacing of individual dosages of a Compound represented by Formula (I) will be determined by the nature and extent of the condition being treated, the form, route and site of administration, and the particular patient being treated, and that such optimums can be determined by conventional techniques. Similarly, the optimal course of treatment, i.e., the number of doses of a Compound represented by Formula (I) given per day for a defined number of days, can be ascertained by those skilled in the art using conventional course of treatment determination tests. Exemplary daily dosage regimens may include from about 0.05 to about 100 mg/kilogram of total body weight, from about 0.1 to about 80 mg/kilogram of total body weight, or from about 0.5 to about 50 mg/kilogram of total body weight, or from about 1 to about 10 mg/kg of total body weight.

Methods of treatment using a Compound represented by Formula (I) may also include dosage regimens that occur less than on a daily basis, for example, several times a week, bi-weekly, weekly, bimonthly, or monthly. Additional treatments may include long-term injectables including, for example, monthly injectables. Term of dosage regimens for the method of the present invention are dependent of a variety of factors include, for example, the objectives of the therapy and health of the patient. Exemplary terms of dosage regimens for the method of the present invention include, for example, from one treatment to treatment that extend for 15 years, one treatment up to treatments extending for 6 years, or treatments lasting from 3 months up to 3 years. The dosage regimen may also include a lifetime maintenance dosage in accordance with the exemplary dosages noted herein.

A bolus administered over a short time once a day is a convenient dosing schedule. Alternatively, the daily dose may be divided into multiple doses for purposes of administration, for example, two to twelve doses per day. Dosage levels of active ingredients in a pharmaceutical composition can also be varied so as to achieve a transient or sustained concentration of the compound in a subject, especially in and around the site of carcinogenesis, and to result in the desired response.

Administration of the formulations of the present invention may also be by an initial dose of a Compound represented by Formula (I) at a level lower than required to achieve the desired effect and to gradually increase the dosage until the desired effect is achieved. It will be understood that the specific dose level for any particular subject will depend on a variety of factors, including body weight, general health, diet, natural history of disease, route and scheduling of administration, combination with one or more other drugs, and severity of disease.

When a Compound represented by Formula (I) is used in combination with other therapeutic agents, the ratio of the Compound represented by Formula (I) to the other therapeutic agent will be varied as needed according to the desired therapeutic effect, the observed side-effects of the combination, or other such considerations known to those of ordinary skill in the medical arts. For example, the ratio of the Compound represented by Formula (I) to other therapeutic agents (e.g., potentiating agents and/or a chemotherapeutic agent) may include a range from about 0.5 to 99.5 wt. %, 1 to 50 wt. % or 1 to 20 wt. % of the Compound represented by Formula (I).

When a Compound represented by Formula (I) is administered before or after other therapeutic agents to treat cancer or other diseases, the respective doses and the dosing regimen of the Compound represented by Formula (I) and the other therapeutic agent may vary. The adjunct or combination therapy can be sequential, that is, the treatment with one agent first and then the second agent, or it can be concomitant treatment wherein two or more agents are administered substantially at the same time. The sequential therapy can be within a reasonable time after the completion of the first therapy before beginning the second therapy. The treatment with both agents at the same time can be in the same daily dose or in separate doses. For example, treatment will be with one agent on day 1 and the other on day 2. The exact regimen will depend on the disease being treated, the severity of the disease and the response to the treatment.

Without requiring a particular mechanism of action, treatment with a Compound represented by Formula (I) may or may not cause the death by apoptosis of cancer cells. With regard to colon cancer, this treatment may also restore a healthy balance between proliferation and apoptosis in the subject's population of enterocytes.

A kit may be provide for treating cancer comprising a Compound represented by Formula I and instructions for a dosage regimen. In addition, the kit may comprising discrete quantities of the compound as well as notes/recommendations on how to administer the compound for the treatment of a certain cancer or cancers, for example those noted hereinabove.

In addition, administration of a Compound represented by Formula (I) might also inhibit production of cytokines and growth factors that are important for sustained growth and progression of cancers.

### EXAMPLES

Without further elaboration, it is believed that one skilled in the art can, using the preceding description, utilize the present invention to its fullest extent. The following examples are, therefore, to be construed as merely illustrative and not a limitation of the scope of the present invention in any way.

### Example 1

### Inhibition of T84 and CaCo-2 Colon Carcinoma Cell Proliferation

The effect of N,N,-diethyl-8,8-dipropyl-2-azaspiro[4,5]decane-2-propanamine dimaleate (Compound 1) on proliferation of human colon carcinoma cells T84 and CaCo-2 was evaluated in the following manner. Cell proliferation was measured by WST-1 dye conversion to Formazan assay using the proliferation kit from BioVision, CA. The procedure used was essentially as described in the manufacture's instructions. Briefly, cells were grown for 7 days until they formed semi-confluent monolayers. On day 7, cells were trypsinized and resuspended in 96-well plates at a concentration of approximately 40,000 cells/well and allowed to grow for 24 hours at 37° C.

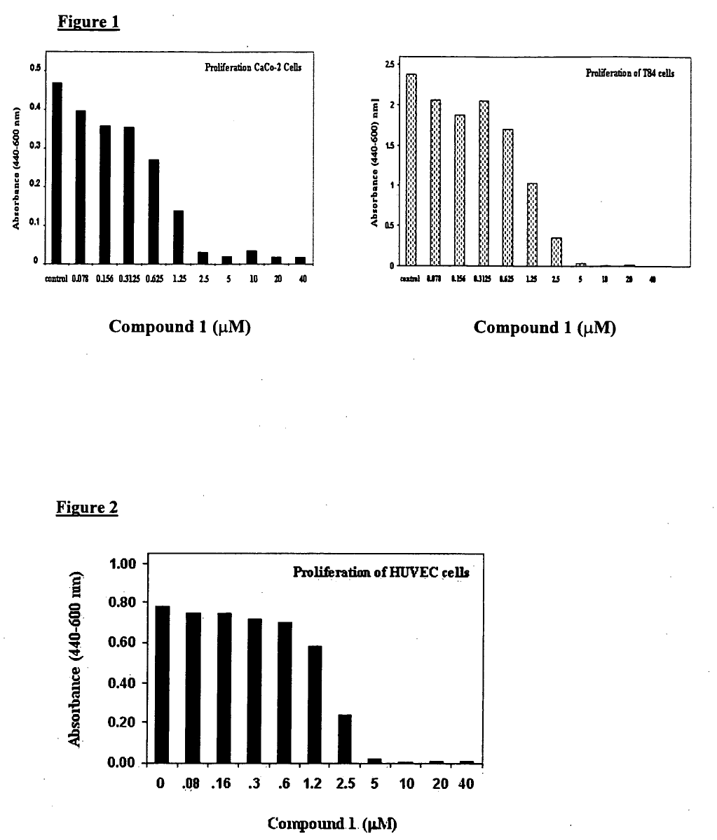

Subsequently, fresh media containing increasing concentrations of Compound 1, as indicated in the FIG. 1, were added and assay plates were further incubated for an additional period of 24 hours. A solution of 5 μl of WST-1 dye per well was added and plates were read after 4 hours at 440 and 600 nm using an ELISA plate reader. The absorbance at 440 minus that at 600 nm is directly proportion to the number of proliferating cells. All samples were measured in triplicate and results were expressed as an average of three determinations.

As shown in FIG. 1, Compound 1 inhibited proliferation of both CaCo-2 and T84 cells with IC₅₀ values in the range of 0.625 to 1.25 μM.

### Example 2

### Inhibition of Human Umbilical Vein Endothelial Cell (HUVEC) Proliferation

Endothelial cell proliferation, migration and apoptosis are essential components of the angiogenic process. Hence, we evaluated the effect of N,N,-diethyl-8,8-dipropyl-2-azaspiro[4,5]decane-2-propanamine dimaleate (Compound 1) on proliferation and apoptosis of HUVEC cells using the same procedures as described for T84 and CaCo-2 cells in Example 1.

As shown in FIG. 2, Compound I inhibited proliferation of HUVEC with an IC₅₀ value in the range of 1.25 to 2.5 μM.

### Example 3

### Induction of Apoptosis in CaCo-2, T84 and HUVEC Cells

T84, CaCo-2 and HUVEC cells, respectively, were grown in 100 mm dishes for 7-9 days, culture media for HUVEC was EGM-2 (Clonetics, BioWhitaker Co.), until they reached semi-confluency. Cell monolayers were then treated for 12 hours (CaCo-2 and T84) and 16 hours (HUVEC), respectively, at the indicated concentrations of N,N,-diethyl-8,8-dipropyl-2-azaspiro[4,5]decane-2-propanamine dimaleate (Compound 1) in culture media. Cells were collected by trypsinization and the apoptotic DNA was isolated from these cells following the instructions of the DNA fragmentation analysis kit (Boehringer Mannheim Corp., Indianapolis, Ind.). The apoptotic DNA was evaluated using 1.5% agarose gel electrophoresis followed by staining with ethidium bromide. M denotes the lane containing molecular weight markers of DNA.

As shown in FIG. 3, treatment of CaCo-2 and T84 cells, respectively, with Compound 1 resulted in formation of DNA laddering in a dose-dependent manner. DNA ladder formation is a well-established hallmark of cells undergoing apoptosis. See: Reed, J. C. Mechanisms of apoptosis avoidance in cancer. Current. Opin. Oncology 11: 68-75, 1999. Seymore, M. Colorectal cancer: Treatment of advanced disease. Cancer Treat. Rev., 24: 119-131, 1998. Wyllie, A. H. Apoptosis and carcinogenesis. Eur. J. Cell Biol., 73: 189-197, 1997. Naik, P., Karrim, J., and Hanahan, D. The rise and fall of apoptosis during multistage tumorigenesis: down modulation contributes to tumor progression from angiogenic progenitors. Genes & Dev. 10: 2105-2116, 1996. The formation of a DNA ladder is commonly observed when cancer cells are treated with pro-apoptotic and anti-cancer compounds. See: Pasricha P. J., Bedi., A., O'Connor K., Rashid, A., Akhtar, A. J., Zahurak, M. L., Piantadosi, S., Hamilton, S. R., and Giardiello, F. M. The effects of sulindac on colorectal proliferation and apoptosis in familial adenomatous polyposis. Gastroenterology 109:994-998, 1995. Thompson W J, Piazza G A, Li H, Liu L, Fetter J, Zhu B, Sperl G, Ahnen D, Pamukcu R. Exisulind induction of apoptosis involves guanosine 3′,5′-cyclic monophosphate phosphodiesterase inhibition, protein kinase G activation, and attenuated beta-catenin. Cancer Res. 60:3338-3342, 2000. Rice P L, Goldberg R J, Ray E C, Driggers L J, Ahnen D J. Inhibition of extracellular signal-regulated kinase 1/2 phosphorylation and induction of apoptosis by sulindac metabolites. Cancer Res. 61:1541-1547, 2001. Hughes, F. M. Jr., and Cidlowski, J. A. Potassium is a critical regulator of apoptotic enzymes in vitro and in vivo. Adv. Enzyme Regul., 39:157-171, 1999.

Initiation of apoptosis in T84 cells was observed at the concentration range of 0.5 to 1 μM. The same extent of apoptosis was achieved in CaCo-2 cells at a concentration range of 1.5 to 2.0 μM.

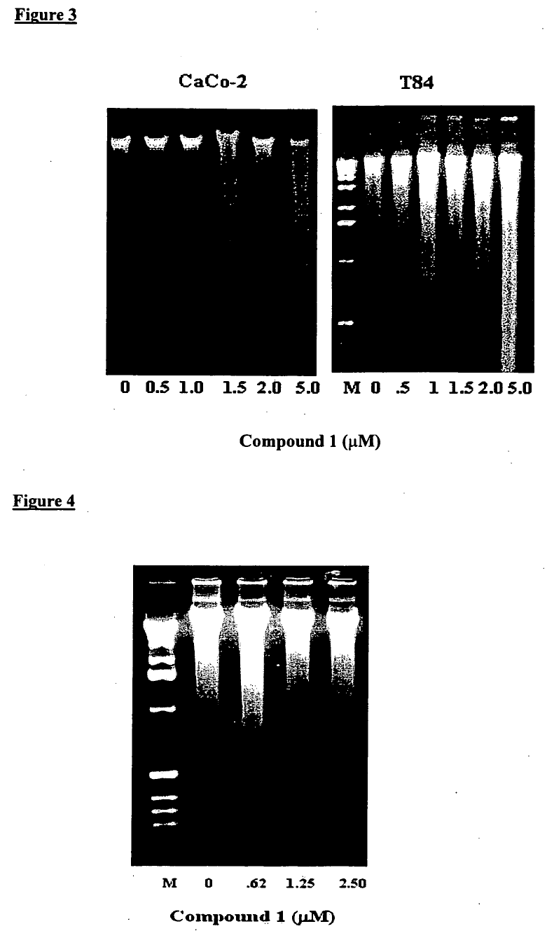

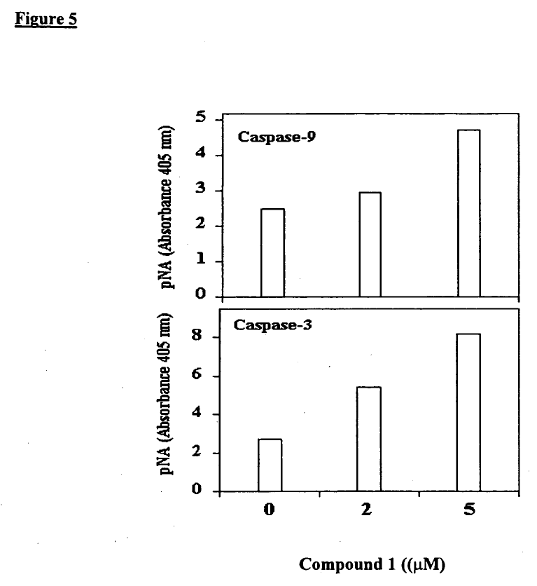

FIG. 4 shows that treatment of HUVEC cells with Compound 1 also resulted in the formation of DNA laddering in a dose-dependent manner, with initiation of apoptosis being achieved at concentrations of Compound 1 somewhere between 0.625 to 1.25 μM.

### Example 4

### Activation of Caspases in T84 Colon Carcinoma Cells

Activities of caspase-3 and caspase-9 were measured using colorimetric assay kits (BioVision, CA). The procedure used was essentially the same as described in the manufacturer's instruction. Briefly, 7-day-old monolayers of T 84 cells in 100 mm dishes were treated with either vehicle (as control) or N,N,-diethyl-8,8-dipropyl-2-azaspiro[4,5]decane-2-propanamine dimaleate (Compound 1) at the indicated respective concentrations for 10 hours. After the treatment, cells were washed with PBS and the cell extracts were prepared by resuspending cells in 200 μl (˜10⁸ cells) of lysis buffer provided in the kit. Cell debris was removed by centrifugation at 10,000×g for 30 min. Supernatants (50-100 μg of protein) were pre-incubated with 10 mM dithiothreitol, 50 mM HEPES, 10% sucrose, 0.1% CHAPS (pH 7.5) and the reaction was started with the addition of 100 μM of the appropriate substrate (DEVD-pNA for caspase-3 and LEHD-pNA for caspase-9). The assay plates (96-well) were incubated at 37° C. for 2 hours, and the yellow color resulting from the release of pNA was measured at 405 nm using an ELISA reader. Samples were run in triplicate and results were expressed as an average of three determinations.

### Example 5

### In Vitro Measurement of Anti-Tumor Effects on Various Cancer Cell Lines

An in vitro assay which tested the ability of N,N,-diethyl-8,8-dipropyl-2-azaspiro[4,5]decane-2-propanamine dimaleate (Compound 1) to inhibit the growth of known cancer cell lines was performed by the National Cancer Institute in accordance with standard procedures. See: Lin, Z. X., Hoult, J. R., and Raman, A. Sulphorhodamine B assay for measuring proliferation of a pigmented melanocyte cell line and its application to the evaluation of crude drugs used in the treatment of vitiligo. J Ethnopharmacol. 66: 141-150, 1999. Briefly, selected tumor cell lines were cultured in media containing five different concentrations of Compound 1. After 48 hours of continuous exposure, a sulforhodamine B (SRB) assay was used to estimate cell viability or growth via optical measurements. These data are reported as follows: Tables 1a and 1b show the optical density as a function of Compound 1 concentration.

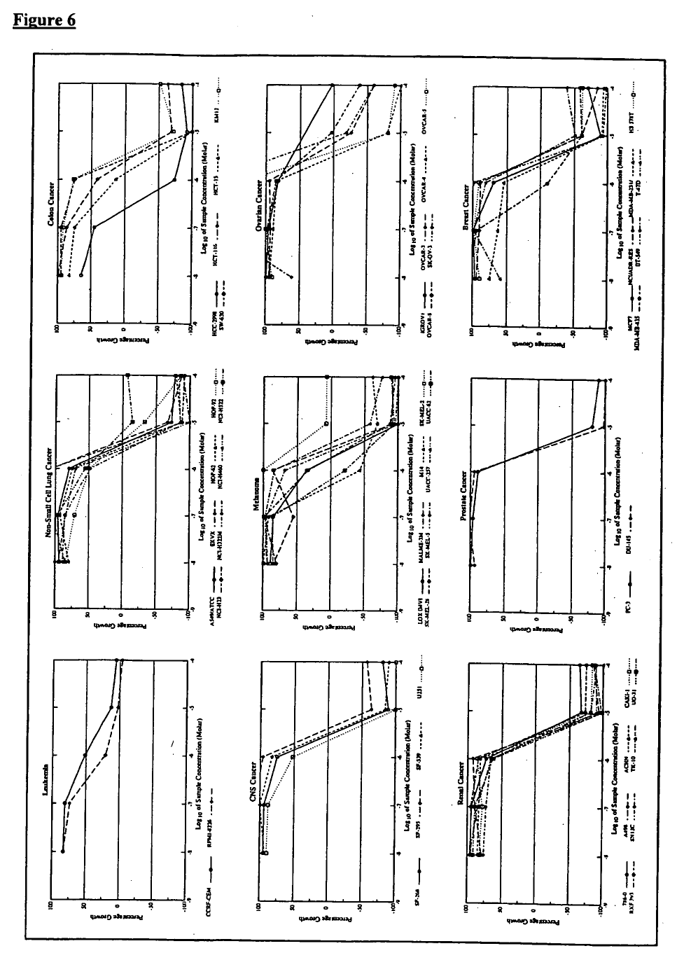

From the measurements, the following values were determined: 1) the concentration of Compound 1 at which a tumor cell growth inhibition of 50% (relative to control) occurs (GI50), 2) the concentration of Compound 1 at which no growth occurs (total growth inhibition, TGI), and 3) the concentration of Compound 1 at which the tumor cell density is half of the control (LC 50). Table 2 reports these data in Log₁₀. FIG. 6 illustrates, graphically, tumor cell growth as a function of Compound 1 concentration of the scanned tumor cell lines.

TABLE 1a
In Vitro Testing Results Showing GI50, TGI, and LC50 of Compound 1 Against Various Tumor Lines
LOG 10 Concentration*

| | Time | | Mean Optical Densities | | | | | Percent Growth | | | | | | | |
|---|---|---|---|---|---|---|---|---|---|---|---|---|---|---|---|
| Panel/Cell Line | Zero | Ctrl | −8.0 | −7.0 | −6.0 | −5.0 | −4.0 | −8.0 | −7.0 | −6.0 | −5.0 | −4.0 | GI50 | TGI | LC50 |

Leukemia

| | | | | | | | | | | | | | | | |
|---|---|---|---|---|---|---|---|---|---|---|---|---|---|---|---|
| CCRF-CEM | 0.076 | 0.723 | 0.611 | 0.597 | 0.409 | 0.158 | 0.115 | 83 | 81 | 51 | 13 | 6 | 1.09E−06 | >1.00E−04 | >1.00E−04 |
| RPMI-8226 | 0.191 | 1.068 | 0.918 | 0.843 | 0.371 | 0.218 | 0.185 | 83 | 74 | 21 | 3 | −3 | 2.83E−07 | 2.98E−05 | >1.00E−04 |

Non-Small Cell Lung Cancer

| | | | | | | | | | | | | | | | |
|---|---|---|---|---|---|---|---|---|---|---|---|---|---|---|---|
| A549/ATCC | 0.256 | 1.346 | 1.277 | 1.307 | 1.126 | 0.070 | 0.056 | 94 | 96 | 80 | −73 | −78 | 1.57E−06 | 3.33E−06 | 7.08E−06 |
| EKVX | 0.619 | 1.281 | 1.280 | 1.336 | 1.321 | 0.202 | 0.073 | 100 | 108 | 106 | −67 | −88 | 2.11E−06 | 4.09E−06 | 7.94E−06 |
| HOP-62 | 0.418 | 0.783 | 0.711 | 0.757 | 0.693 | 0.056 | 0.090 | 80 | 93 | 75 | −87 | −79 | 1.43E−06 | 2.91E−06 | 5.93E−06 |
| HOP-92 | 0.815 | 1.656 | 1.529 | 1.412 | 1.227 | 0.542 | 0.106 | 85 | 71 | 49 | −33 | −87 | 8.97E−07 | 3.92E−06 | 2.03E−05 |
| NCI-H23 | 0.368 | 0.791 | 0.728 | 0.814 | 0.822 | 0.040 | 0.030 | 85 | 105 | 107 | −89 | −92 | 1.96E−06 | 3.52E−06 | 6.32E−06 |
| NCI-H322M | 0.697 | 1.026 | 0.991 | 0.979 | 0.855 | 0.100 | 0.115 | 89 | 86 | 48 | −86 | −84 | 8.90E−07 | 2.29E−06 | 5.41E−06 |
| NCI-H460 | 0.139 | 1.474 | 1.403 | 1.278 | 1.075 | −0.001 | 0.015 | 95 | 85 | 70 | −100 | −90 | 1.31E−06 | 2.58E−06 | 5.08E−06 |
| NCI-H522 | 0.107 | 0.542 | 0.518 | 0.531 | 0.347 | 0.091 | 0.099 | 94 | 97 | 55 | −15 | −7 | 1.18E−06 | 6.05E−06 | >1.00E−04 |

Colon Cancer

| | | | | | | | | | | | | | | | |
|---|---|---|---|---|---|---|---|---|---|---|---|---|---|---|---|
| HCC-2998 | 0.220 | 0.404 | 0.338 | 0.303 | 0.057 | 0.019 | 0.036 | 64 | 45 | −74 | −92 | −84 | 5.49E−08 | 2.39E−07 | 6.28E−07 |
| HCT-116 | 0.163 | 0.752 | 0.744 | 0.675 | 0.407 | 0.051 | 0.061 | 99 | 87 | 41 | −69 | −63 | 6.46E−07 | 2.37E−06 | 6.73E−06 |
| HCT-15 | 0.265 | 1.000 | 0.878 | 0.819 | 0.371 | −0.027 | −0.026 | 83 | 75 | 14 | −100 | −100 | 2.61E−07 | 1.34E−06 | 3.65E−06 |
| KM12 | 0.436 | 1.487 | 1.429 | 1.402 | 1.243 | 0.123 | 0.210 | 94 | 92 | 77 | −72 | −52 | 1.51E−06 | 3.29E−06 | 7.13E−06 |
| SW-620 | 0.212 | 0.986 | 0.948 | 0.948 | 0.789 | −0.002 | −0.016 | 95 | 95 | 75 | −100 | −100 | 1.38E−06 | 2.67E−06 | 5.17E−06 |

CNS Cancer

| | | | | | | | | | | | | | | | |
|---|---|---|---|---|---|---|---|---|---|---|---|---|---|---|---|
| SF-268 | 0.295 | 1.236 | 1.193 | 1.181 | 1.004 | 0.033 | 0.058 | 95 | 94 | 75 | −89 | −80 | 1.43E−06 | 2.88E−06 | 5.80E−06 |
| SF-295 | 0.554 | 1.565 | 1.614 | 1.653 | 1.520 | 0.202 | 0.239 | 105 | 109 | 96 | −64 | −57 | 1.93E−06 | 3.98E−06 | 8.21E−06 |
| SF-539 | 0.309 | 0.612 | 0.591 | 0.612 | 0.559 | 0.050 | 0.033 | 93 | 100 | 83 | −84 | −89 | 1.57E−06 | 3.13E−06 | 6.25E−06 |
| U251 | 0.218 | 0.955 | 0.885 | 0.864 | 0.602 | −0.049 | −0.042 | 90 | 88 | 52 | −100 | −100 | 1.03E−06 | 2.20E−06 | 4.69E−06 |

Melanoma

| | | | | | | | | | | | | | | | |
|---|---|---|---|---|---|---|---|---|---|---|---|---|---|---|---|
| LOX IMVI | 0.271 | 1.174 | 1.072 | 1.037 | 0.595 | −0.044 | −0.030 | 89 | 85 | 36 | −100 | −100 | 5.15E−07 | 1.84E−06 | 4.29E−06 |
| MALME-3M | 0.938 | 1.135 | 1.103 | 1.047 | 1.108 | 0.089 | 0.084 | 84 | 55 | 86 | −91 | −91 | 1.60E−06 | 3.07E−06 | 5.90E−06 |
| M14 | 0.279 | 0.889 | 0.884 | 0.838 | 0.158 | 0.086 | 0.109 | 99 | 92 | −43 | −69 | −61 | 2.03E−07 | 4.77E−07 | 1.80E−06 |
| SK-MEL-2 | 0.086 | 1.203 | 1.191 | 1.191 | 1.199 | 0.167 | 0.168 | 99 | 99 | 100 | 7 | 7 | 3.45E−06 | >1.00E−04 | >1.00E−04 |
| SK-MEL-28 | 0.515 | 1.492 | 1.301 | 1.473 | 0.846 | 0.061 | 0.026 | 80 | 98 | 34 | −88 | −95 | 5.61E−07 | 1.89E−06 | 4.86E−06 |
| SK-MEL-5 | 0.484 | 1.869 | 1.779 | 1.797 | 1.420 | 0.021 | −0.001 | 93 | 95 | 68 | −96 | −100 | 1.28E−06 | 2.59E−06 | 5.25E−06 |
| UACC-257 | 0.455 | 1.459 | 1.403 | 1.427 | 1.302 | 0.190 | 0.113 | 94 | 97 | 84 | −58 | −75 | 1.74E−06 | 3.90E−06 | 8.74E−06 |
| UACC-62 | 0.529 | 1.518 | 1.375 | 1.379 | 0.421 | 0.034 | 0.060 | 85 | 86 | −21 | −94 | −89 | 2.18E−07 | 6.42E−07 | 2.53E−06 |

*Concentration expressed in Moles/L

TABLE 1b
In Vitro Testing Results Showing GI50, TGI, and LC50 of Compound 1 Against Various Tumor Lines
Log10 Concentration*

| | Time | | Mean Optical Densities | | | | | Percent Growth | | | | | | | |
|---|---|---|---|---|---|---|---|---|---|---|---|---|---|---|---|
| Panel/Cell Line | Zero | Ctrl | −8.0 | −7.0 | −6.0 | −5.0 | −4.0 | −8.0 | −7.0 | −6.0 | −5.0 | −4.0 | GI50 | TGI | LC50 |

Renal Cancer

| | | | | | | | | | | | | | | | |
|---|---|---|---|---|---|---|---|---|---|---|---|---|---|---|---|
| 786-0 | 0.365 | 1.139 | 1.111 | 1.091 | 0.934 | 0.121 | 0.132 | 96 | 94 | 73 | −67 | −64 | 1.47E−06 | 3.33E−06 | 7.57E−06 |
| A498 | 1.067 | 1.359 | 1.297 | 1.327 | 1.336 | 0.065 | 0.157 | 79 | 89 | 92 | −94 | −85 | 1.68E−06 | 3.13E−06 | 5.81E−06 |
| ACHN | 0.323 | 0.792 | 0.721 | 0.711 | 0.705 | −0.031 | −0.041 | 85 | 83 | 81 | −100 | −100 | 1.49E−06 | 2.81E−06 | 5.30E−06 |
| CAKI-1 | 0.800 | 1.832 | 1.646 | 1.607 | 1.691 | 0.150 | 0.136 | 82 | 78 | 86 | −81 | −83 | 1.65E−06 | 3.27E−06 | 6.51E−06 |
| RXF 393 | 0.320 | 1.065 | 1.017 | 1.028 | 0.943 | 0.086 | 0.083 | 94 | 95 | 84 | −73 | −74 | 1.64E−06 | 3.41E−06 | 7.10E−06 |
| SN12C | 0.360 | 0.826 | 0.717 | 0.701 | 0.668 | 0.069 | 0.036 | 76 | 73 | 66 | −81 | −90 | 1.29E−06 | 2.82E−06 | 6.17E−06 |
| TK-10 | 0.909 | 1.472 | 1.449 | 1.389 | 1.283 | 0.087 | 0.101 | 96 | 85 | 66 | −90 | −89 | 1.27E−06 | 2.65E−06 | 5.52E−06 |
| UO-31 | 0.298 | 1.153 | 1.076 | 1.056 | 0.833 | 0.006 | 0.039 | 91 | 89 | 63 | −98 | −87 | 1.20E−06 | 2.45E−06 | 5.03E−06 |

Prostate Cancer

| | | | | | | | | | | | | | | | |
|---|---|---|---|---|---|---|---|---|---|---|---|---|---|---|---|
| PC-3 | 0.356 | 2.141 | 2.116 | 2.067 | 1.953 | 0.073 | 0.037 | 99 | 96 | 89 | −80 | −90 | 1.71E−06 | 3.38E−06 | 6.68E−06 |
| DU-145 | 0.237 | 0.763 | 0.728 | 0.766 | 0.733 | 0.005 | −0.032 | 93 | 101 | 94 | −98 | −100 | 1.70E−06 | 3.09E−06 | 5.62E−06 |

Breast Cancer

| | | | | | | | | | | | | | | | |
|---|---|---|---|---|---|---|---|---|---|---|---|---|---|---|---|
| MCF7 | 0.170 | 1.355 | 1.311 | 1.339 | 0.996 | 0.017 | 0.051 | 96 | 99 | 70 | −90 | −70 | 1.33E−06 | 2.73E−06 | 5.60E−06 |
| NCI/ADR-RES | 0.528 | 1.301 | 1.254 | 1.227 | 1.220 | 0.208 | 0.081 | 94 | 90 | 89 | −61 | −85 | 1.83E−06 | 3.95E−06 | 8.50E−06 |
| MDA-MB-231/ATCC | 0.674 | 0.972 | 0.897 | 0.863 | 0.839 | 0.053 | 0.038 | 75 | 63 | 55 | −92 | −94 | 1.09E−06 | 2.37E−06 | 5.18E−06 |
| HS 578T | 0.693 | 1.270 | 1.206 | 1.258 | 1.217 | 0.269 | 0.264 | 89 | 98 | 91 | −61 | −62 | 1.86E−06 | 3.96E−06 | 8.43E−06 |
| MDA-MB-435 | 0.584 | 1.574 | 1.613 | 1.517 | 0.528 | 0.230 | 0.244 | 104 | 94 | −10 | −61 | −58 | 2.67E−07 | 8.07E−07 | 6.19E−06 |
| BT-549 | 0.459 | 0.865 | 0.859 | 0.904 | 0.856 | 0.027 | 0.007 | 98 | 109 | 98 | −94 | −98 | 1.77E−06 | 3.23E−06 | 5.89E−06 |
| T-47D | 0.332 | 1.191 | 0.837 | 1.176 | 1.024 | 0.164 | 0.207 | 59 | 98 | 81 | −51 | −38 | 1.71E−06 | 4.11E−06 | |

*Concentration expressed in Moles/L

TABLE 2
Log₁₀ Values of GI50, TGI, and LC50 of Compound 1
for Various Tumor Cell Lines

| Panel/Cell Line | Log₁₀ GI50 | | Log₁₀ TGI | | Log₁₀ LC50 |
|---|---|---|---|---|---|
| Leukemia | | | | | |
| CCRF-CEM | −5.96 | > | −4.00 | > | −4.00 |
| RPMI-8226 | −6.55 | | −4.53 | > | −4.00 |
| Non-Small Cell | | | | | |
| Lung Cancer | | | | | |
| A549/ATCC | −5.80 | | −5.48 | | −5.15 |
| EKVX | −5.68 | | −5.39 | | −5.10 |
| HOP-62 | −5.84 | | −5.54 | | −5.23 |
| HOP-92 | −6.05 | | −5.41 | | −4.69 |
| NCI-H23 | −5.71 | | −5.45 | | −5.20 |
| NCI-H322M | −6.05 | | −5.64 | | −5.27 |
| NCI-H460 | −5.88 | | −5.59 | | −5.29 |
| NCI-H522 | −5.93 | | −5.22 | > | −4.00 |
| Colon Cancer | | | | | |
| HCC-2998 | −7.26 | | −6.62 | | −6.20 |
| HCT-116 | −6.19 | | −5.63 | | −5.17 |
| HCT-15 | −6.58 | | −5.87 | | −5.44 |
| KM12 | −5.82 | | −5.48 | | −5.15 |
| SW-620 | −5.86 | | −5.57 | | −5.29 |
| CNS Cancer | | | | | |
| SF-268 | −5.84 | | −5.54 | | −5.24 |
| SF-295 | −5.71 | | −5.40 | | −5.09 |
| SF-539 | −5.80 | | −5.50 | | −5.20 |
| U251 | −5.99 | | −5.66 | | −5.33 |
| Melanoma | | | | | |
| LOX IMVI | −6.29 | | −5.74 | | −5.37 |
| MALME-3M | −5.80 | | −5.51 | | −5.23 |
| M14 | −6.69 | | −6.32 | | −5.74 |
| SK-MEL-2 | −5.46 | > | −4.00 | > | −4.00 |
| SK-MEL-28 | −6.25 | | −5.72 | | −5.31 |
| SK-MEL-5 | −5.89 | | −5.59 | | −5.28 |
| UACC-257 | −5.76 | | −5.41 | | −5.06 |
| UACC-62 | −6.66 | | −6.19 | | −5.60 |
| Ovarian Cancer | | | | | |
| IGROV1 | −5.61 | > | −4.00 | > | −4.00 |
| OVCAR-3 | −5.63 | | −5.21 | | −4.29 |
| OVCAR-4 | −5.79 | | −5.49 | | −5.18 |
| OVCAR-5 | −5.62 | | −5.39 | | −5.15 |
| OVCAR-8 | −5.57 | | −4.92 | > | −4.00 |
| SK-OV-3 | −5.34 | | −5.10 | | −4.23 |
| Renal Cancer | | | | | |
| 786-0 | −5.83 | | −5.48 | | −5.12 |
| A498 | −5.77 | | −5.50 | | −5.24 |
| ACHN | −5.83 | | −5.55 | | −5.28 |
| CAKI-1 | −5.78 | | −5.49 | | −5.19 |
| RXF 393 | −5.79 | | −5.47 | | −5.15 |
| SN12C | −5.89 | | −5.55 | | −5.21 |
| TK-10 | −5.90 | | −5.58 | | −5.26 |
| UO-31 | −5.92 | | −5.61 | | −5.30 |
| Prostate Cancer | | | | | |
| PC-3 | −5.77 | | −5.47 | | −5.18 |
| DU-145 | −5.77 | | −5.51 | | −5.25 |
| Breast Cancer | | | | | |
| MCF7 | −5.88 | | −5.56 | | −5.25 |
| NCI/ADR-RES | −5.74 | | −5.40 | | −5.07 |
| MDA-MB- | −5.96 | | −5.63 | | −5.29 |
| 231/ATCC | −5.73 | | −5.40 | | −5.07 |
| HS 578T | −6.57 | | −6.09 | | −5.21 |
| MDA-MB-435 | −5.75 | | −5.49 | | −5.23 |
| BT-549 | −5.77 | | −5.39 | | |
| T-47D | | | | | |
| MG_MID | −5.93 | | −5.45 | | −5.06 |
| Delta | 1.33 | | 1.18 | | 1.15 |
| Range | 1.92 | | 2.62 | | 2.20 |

### Example 6

### Anti-Angiogenesis CAM Assay

White Leghorn eggs, incubated for 10 days, were dosed with the amounts of Compound 1 as indicated in Table 3. The dosing was effected by pipetting 40 μl of the indicated solution onto a 13 mm round Thermanox® coverslip and allowing it to air dry. After the material appeared dry, the coverslip was placed onto the chorioallantoic membrane (CAM) of each egg insuring contact of the dried test article with the CAM. After dosing, the eggs were returned to the incubator for approximately 48 hours. Following the 48 hour exposure period, the eggs were removed from the incubator, observed for viability, and the exposed area under the coverslip was examined for loss of vasculature. The data from this experiment are reported in Table 3.

TABLE 3
CAM Assay Showing Anti-angiogenic Data for Compound 1*

| | Dose | | | | | | |
|---|---|---|---|---|---|---|---|
| | Per | | | | | | |
| | Egg | Dead | % Blood Vessel Clearance | | | | |
| Test Solution | (μg) | Eggs | 0 | <25 | <50 | <75 | >75 |
| Thalidomide | 100 | 0 | 2 | 3 | 3 | 2 | |
| Compound 1 (0 mg/ml) | 0 | 1 | 19 | | | | |
| Compound 1 | 25 | 0 | 8 | 2 | | | |
| (0.625 mg/ml) | | | | | | | |
| Compound 1 | 50 | 0 | 5 | 4 | 1 | | |
| (1.25 mg/ml) | | | | | | | |
| Compound 1 (2.5 mg/ml) | 100 | 4 | 1 | 1 | 3 | 1 | |
| Compound 1 (5 mg/ml) | 200 | 3 | 0 | 2 | 5 | | |
| Compound 1 (10 mg/ml) | 400 | 7 | 0 | 1 | 0 | 2 | |

*Twenty eggs were used in the control group and ten eggs were used in each of the treatment groups. The inhibition in angiogenesis is based on the visual observation of the loss of generation of new blood vessels in the area under the cover slips.

### Example 7

### In Vivo Administration of Compound 1 to Live Rats

[¹⁴C] N,N,-diethyl-8,8-dipropyl-2-azaspiro[4,5]decane-2-propanamine dimaleate salt, (“Compound II”) with a specific activity of 91 uCi/mg and chemical purity >99%, and non-radiolabeled dimaleate salt of N,N,-diethyl-8,8-dipropyl-2-azaspiro[4,5]decane-2-propanamine dimaleate (Compound 1) chemical purity >98%, respectively, were used in this study. The site of the radiocarbon label in Compound II is depicted by an asterisk in Formula II. The non-radiolabeled material was used for reference purposes. The radiolabeled material was stored at ca ˜80° C. in the dark and the non-radiolabeled material at ca −20° C. in the dark. The radiochemical purity of Compound II was confirmed by TLC and was found to be 98.0% (60F254 silica gel plate; eluted in dichloromethane: methanol: ammonia 80:18:2; detected with an Isomess IM-3016 radio-TLC analyzer or Phosphor Imager SF).

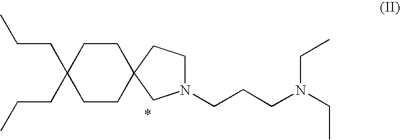

Three healthy male Sprague Dawley rats (Crl:CD(SD)BR), age ca 8-9 weeks, body weights at dosing 237-255 g, were supplied by Charles River (UK) Limited. The animals were housed in holding cages suitable for this species for 8 days prior to use. SDS Rat and Mouse Maintenance Diet No. 1, (Special Diets Services, Witham Essex), and mains quality water were available ad libitum throughout. The diet and water supplied to the animals were routinely analyzed for quality and no problems were detected. Holding and study areas had automatic control of light cycle and temperature. The actual range of temperature measured during the study was 17-25° C. with relative humidity measured at 60%.

The dose was prepared for oral administration on the day of dosing. An appropriate amount of Compound II was dissolved in distilled water to give a target concentration of 1 mg f.b./mL. Following dosing, a radiochemical purity check was conducted on the dose formulation using the TLC method described above, and the radiochemical purity was shown to be >97%. This demonstrated that degradation of the radiolabeled compound during the dosing period was negligible.

For each rat, an aliquot of the dose solution was administered orally by gavage. Doses were administered at a nominal dose volume of 10 mL/kg, using a 5.0 mL glass syringe fitted with a gavage needle. For each dose, the combined weight of dose and dosing equipment were recorded prior to dosing and the discharged dosing equipment was weighed after dosing. The concentration of radioactive material in each dose solution was also determined. From these data the actual doses received by the animals were determined and are shown in Table 4.

TABLE 4
Dosage of Compound II Received by Test Animals

| | | Dose Received | | |
|---|---|---|---|---|
| Animal Number | Animal Weight (kg) | MBq | mg f.b. | mg f.b./kg |
| #1 male | 0.237 | 1.32 | 0.232 | 0.978 |
| #2 male | 0.240 | 1.31 | 0.230 | 0.958 |
| #3 male | 0.255 | 1.41 | 0.247 | 0.969 |

Oral Administration: Target Dose Level 1 mg f.b./kg

Immediately following dosing, the rats were placed into all glass metabolism cages suitable for the quantitative collection of excreta and expired air. All samples were collected into individual, uniquely labeled containers.

Urine was collected during the periods 0-6,6-24, 24-48, 48-72, 72-96, 96-120, 120-144 and 144-168 hours post dose. The collection containers were cooled by solid CO₂ during the first 48 hours after dosing.

Feces were collected for the periods 0-24, 24-48, 48-72, 72-96, 96-120, 120-144 and 144-168 hours post dose. Collection containers were cooled by solid CO₂ during the first 48 hours.

At the time of each feces collection, each cage was washed with water (ca 750 mL) and the washings were retained for radioassay. During the periods 0-24 h and 24-48 hours after dosing, expired air was passed through 2 serial traps containing ethanolamine:ethoxyethaonol (3:7, v/v) in order to effect the removal of CO₂. The trap solvent was sampled for radioassay at the end of each collection period.

At 168 hours post dose, each animal was killed by CO₂ narcosis and cervical dislocation. Gastrointestinal tracts were removed and retained separately from the carcasses in preparation for radioassay.

All urine and feces samples were stored frozen (ca 20°) prior to and after analysis. Cage washes were stored at room temperature until analysis was complete and were then discarded. All carcasses and gastro-intestinal tracts were stored frozen at ca-20° C. prior to analysis.

Duplicate aliquots of urine (ca 0.3 mL) and cage washings (ca 1 mL) were dispensed, diluted to 1 mL with distilled water (if considered necessary) and mixed with Quickzint 1 scintillation fluid (10 mL; Zinsser Analytic Maidenhead, UK).

Feces were homogenised in 1 to 2 volumes of water with the sample and homogenate weight recorded. Duplicate aliquots (ca 0.3) were taken from each sample and dispensed onto combustopads contained in combustocones (Can berra Packard Limited, Pangbourne, UK). When dry, these samples were combusted using a Packard Tri-Carb 306 Automatic Sample Oxidiser. The resultant ¹⁴CO₂ generated was collected by absorption in Carbo-Sorb® (8 mL; Can berra Packard Limited) to which Permafluor®E⁺ scintillation fluid (10 mL; Can berra Packard Limited) was added.

Combustion of standards (Spec-Chec™-¹⁴ C; Canberra Packard Limited) showed that recovery efficiencies were in excess of 97% throughout so the results were used directly and were not corrected for % efficiency.

For each rat, the carcass and gastro-intestinal tract with contents were solubilised in Soluene 350 (Canberra Packard). When dissolved, portions of the digest (ca 0.1 mL) were taken for liquid scintillation spectrometry. The volume was made up to 1 mL by the addition of methanol then 10 mL Quickzint was added and the samples counted as for the other liquid samples.

All samples prepared in scintillant were analyzed for 5 min, together with representative blank and standard vials using a liquid scintillation analyser (Packard Liquid Scintillation Analyser, 1600 TR) with automatic quench correction by external standard ratio. Where possible, samples were analyzed in duplicate and allowed to heat and light stabilized prior to analysis. Prior to calculation of each result, a background count rate was determined and subtracted for each sample count rate. A limit of reliable determination of 30 d.p.m. above background has been instituted in these laboratories.

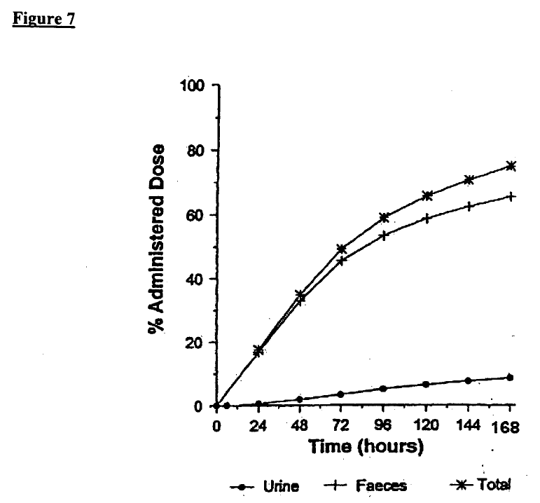

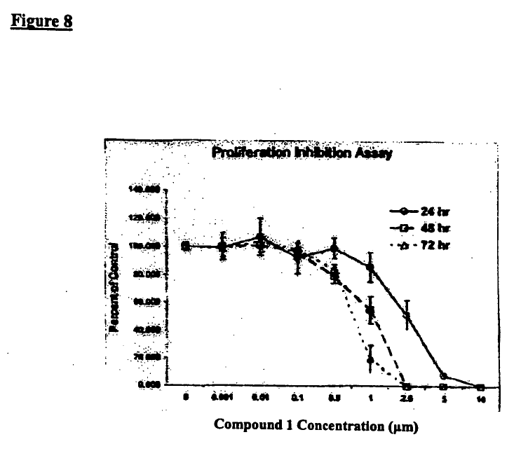

The recovery of total radioactivity for each animal in excreta, gastrointestinal tract and carcass is shown in Tables 5 and 6. Mean excretion results are depicted graphically in FIG. 7.

TABLE 5
Excretion of Radioactivity Following Single Oral Administration of
Compound II to Male rats at a Target Dose Level of 1 mg Free Base/Kg

| Sample and | | | | | |
|---|---|---|---|---|---|
| Collection | | | | | |
| Period (hours) | #1 male | #2 male | #3 male | Mean | SD |
| Urine | | | | | |
| 0-6 | 0.1 | 0.1 | 0.1 | 0.1 | 0.0 |
| 6-24 | 0.4 | 0.5 | 0.5 | 0.5 | 0.1 |
| 24-48 | 1.0 | 1.4 | 1.4 | 1.3 | 0.2 |
| 48-72 | 1.2 | 1.5 | 1.7 | 1.5 | 0.3 |
| 72-96 | 1.3 | 1.6 | 2.0 | 1.6 | 0.4 |
| 96-120 | 1.2 | 1.5 | 1.6 | 1.4 | 0.2 |
| 120-144 | 0.9 | 1.3 | 1.3 | 1.2 | 0.2 |
| 144-162 | 0.8 | 1.0 | 1.2 | 1.0 | 0.2 |
| 0-168 | 6.9 | 8.9 | 9.8 | 8.5 | 1.5 |
| Feces | | | | | |
| 0-24 | 18.2 | 14.7 | 17.7 | 16.9 | 1.9 |
| 24-48 | 16.3 | 17.2 | 14.5 | 16.0 | 1.4 |
| 48-72 | 10.0 | 15.2 | 12.7 | 12.6 | 2.6 |
| 72-96 | 7.5 | 7.8 | 8.0 | 7.8 | 0.3 |
| 96-120 | 5.5 | 4.1 | 6.4 | 5.3 | 1.2 |
| 120-144 | 3.5 | 3.5 | 4.3 | 3.8 | 0.5 |
| 144-168 | 3.0 | 3.2 | 3.1 | 3.1 | 0.1 |
| 0-168 | 64.0 | 65.7 | 66.7 | 65.5 | 1.4 |
| Cage Wash | | | | | |
| 0-24 | 0.1 | 0.1 | *<0.1 | ⁺0.1 | ⁺0.0 |
| 24-48 | 0.1 | <0.1 | 0.1 | 0.1 | 0.1 |
| 48-72 | <0.1 | 0.2 | 0.2 | 0.1 | 0.1 |
| 72-96 | 0.1 | 0.2 | 0.3 | 0.2 | 0.1 |
| 96-120 | 0.1 | 0.2 | 0.2 | 0.2 | 0.1 |
| 120-144 | 0.1 | 0.1 | 0.3 | 0.2 | 0.1 |
| 144-168 | 0.2 | 0.2 | 0.2 | 0.2 | 0.0 |
| 0-168 | 0.7 | 1.0 | 1.3 | 1.0 | 0.3 |

SD = Standard deviation
*= Results calculated from data less than 30 d.p.m. above background
⁺= Value includes results calculated from data less than 30 d.p.m. above background

TABLE 6
Summary of the Recovery of Radioactivity
Following Oral Administration of Compound II
to Male Rats at a Target Dose Level of 1 mg Free Base/Kg

| Sample | #1 male | #2 male | #3 male | Mean | SD |
|---|---|---|---|---|---|
| Urine | 6.9 | 8.9 | 9.8 | 8.5 | 1.5 |
| Feces | 64.0 | 65.7 | 66.7 | 65.5 | 1.4 |
| Cage | 0.7 | 1.0 | *1.3 | ⁺1.0 | ⁺0.3 |
| Washings | | | | | |
| Expired air 1 | *<0.1 | *<0.1 | *<0.1 | ⁺<0.1 | — |
| Expired air 2 | *<0.1 | *<0.1 | *<0.1 | ⁺<0.1 | — |
| GI Tract | 13.8 | 13.3 | 10.5 | 12.5 | 1.8 |
| Carcass | 11.3 | 14.1 | 14.9 | 13.5 | 1.9 |
| Total | 96.7 | 103.0 | 103.2 | 101.0 | 3.7 |

Results expressed as a % administered dose over a 168 hour collection period.
*= Results calculated from data less than 30 d.p.m. above background.
⁺= Value includes results calculated from data less than 30 d.p.m. above background
SD = Standard deviation

The administered radioactive dose was quantitatively recovered (96.7-103.2%). Radioactivity was excreted predominantly in the feces, with a mean of 65.5% of the dose recovered by 168 hours after dosing. In contrast, a mean of 8.5% of the radioactive dose was recovered in the urine by this time. The elimination of radiolabeled material was slow with ca 66% of the dose recovered in the excreta up to 120 hours after dosing. Over the full 168 hour collection period, a mean of 75.0% was recovered in excreta and cage washings with ca 12.5% and 13.5% of the dose remaining in the gastro-intestinal tract and carcass, respectively, at 168 hours. Less than 0.2% of the dose was recovered in expired air.

### Example 8

### Inhibiton of Proliferation of CaCo-2 Cells

The effect of N,N,-diethyl-8,8-dipropyl-2-azaspiro[4,5]decane-2-propanamine dimaleate (Compound 1) and other analogs (structures shown in Table 7) on proliferation of CaCo-2, a human colon carcinoma cell line, were evaluated in the following manner. Cell proliferation was measured by WST-1 dye conversion to Formazan assay using a proliferation kit from BioVision, CA. The procedure used was essentially the same as described in the manufacture's instructions. Briefly, cells were grown for 7 days until they formed semi-confluent monolayers. On day 7, cells were trypsinized and re-suspended in 96-well plates at a concentration of approximately 50,000 cells/well. Subsequently, fresh media containing 5 μM of test compound was added and assay plates were further incubated for an additional period of 12 hours. A solution of 10 μl of WST-1 dye per well was added and plates were read after 4 hours at 440 and 600 nm using an ELISA plate reader. The absorbance at 440-600 nm is directly proportion to the number of proliferating cells. All samples were measured in duplicate and results were expressed as an average of two determinations.

TABLE 7
Inhibition of Proliferation of CaCo-2 Cells*

| | | % Inhibition of | |
|---|---|---|---|
| | | Proliferation at 5 | IC₅₀ |
| Compound | Compound Structure | μM Compound | (μM) |
| Compound 1 | 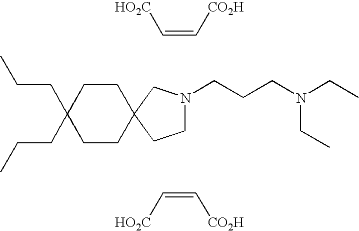 | 85 | ˜2.5 |
| A* | 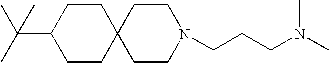 | 45 | ˜20 |
| B* | 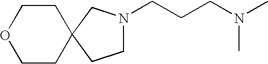 | 0 | >>20 |
| C* | 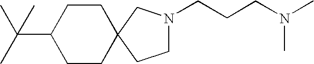 | 15 | >>20 |
| D* | 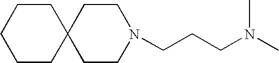 | 0 | >>20 |
| E* | 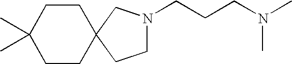 | 0 | >>20 |
| F* | 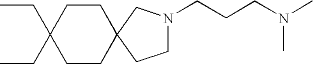 | 35 | >20 |
| G* | 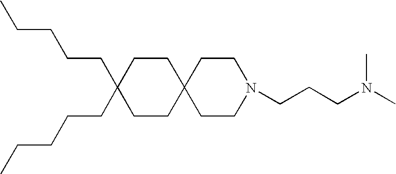 | 86 | ˜2.5 |
| H* | 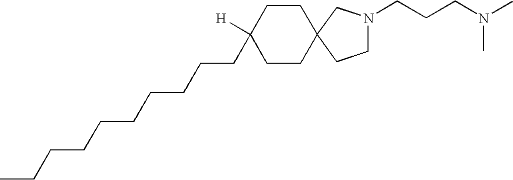 | 73 | ˜3.5 |
| J* | 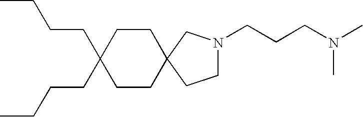 | 84 | ˜2.5 |
| K* | 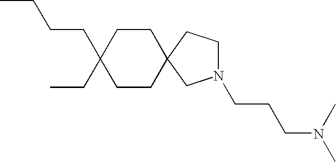 | 81 | ˜2.5 |
| L* | 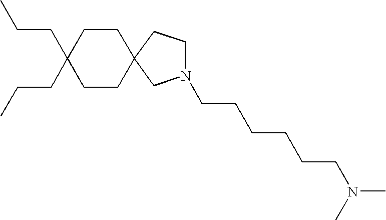 | 76 | ˜3.5 |
| M* | 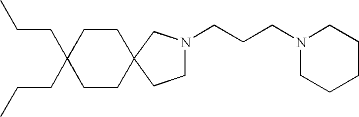 | 67 | ˜4 |

*Note: Compound tested as the dihydrochloride salt form.

### Example 9

### Growth Inhibition Assay

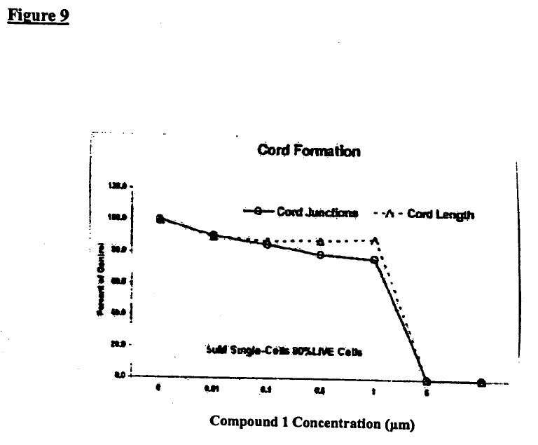

HUVEC (1.5×10³) are plated in a 96-well plate in 100 μl of EBM-2 (Clonetic # CC3162). After 24 hours (day 0), a test solution of Compound 1 (100 μl) is added to each well at 2× the desired concentration (5-7 concentration levels) in EBM-2 medium. On day 0, one plate is stained with 0.5% crystal violet in 20% methanol for 10 minutes, rinsed with water, and air-dried. The remaining plates are incubated for 72 hours at 37° C. After 72 hours, plates are stained with 0.5% crystal violet in 20% methanol, rinsed with water and air-dried. The stain is eluted with 1:1 solution of ethanol: 0.1 M sodium citrate (including day 0 plate), and absorbance is measured at 540 nm with an ELISA reader (Dyriatech Laboratories). Day 0 absorbance is subtracted from the 72 hour plates and data is plotted as percentage of control proliferation (vehicle treated cells). IC₅₀ (drug concentration causing 50% inhibition) was calculated from the plotted data and is reported in Table 8. The plotted data are shown in FIG. 8.

### Example 10

### Cord Formation Assay

Matrigel (60 μl of 10 mg/ml; Collaborative Lab # 35423) was placed in each well of an ice-cold 96-well plate. The plate is allowed to sit at room temperature for 15 minutes then incubated at 37° C. for 30 minutes to permit the matrigel to polymerize. In the mean time, HUVEC are prepared in EGM-2 (Clonetic# CC3162) at a concentration of 2×10⁵ cells/ml. Test solutions of Compound I are prepared at 2× the desired concentration (5 concentration levels) in the same medium. The cells (500 μl) and 2× Compound 1 (500 μl) solution are mixed and 200 μl of this suspension are placed in duplicate on the polymerized matrigel. After a 24 hour incubation, triplicate pictures are taken for each concentration using a Bioquant Image Analysis system. Drug effect (IC₅₀) is assessed compared to untreated controls by measuring the length of cords formed and number of junctions, and is reported in Table 8. The plotted data are shown in FIG. 9.

### Example 11

### Cell Migration Assay

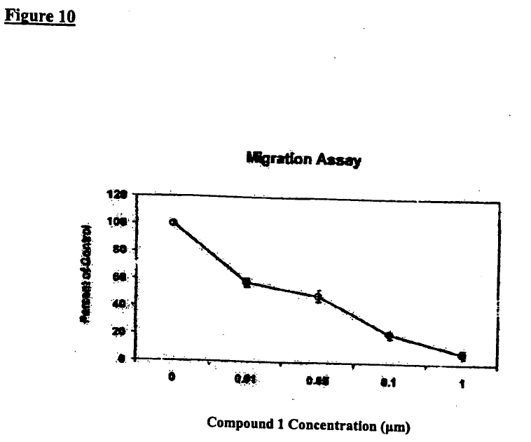

Migration is assessed using the 48-well Boyden chamber and 8 μm pore size collagen-coated (10 μg/ml rat tail collagen; Collaborative Laboratories) polycarbonate filters (Osmonics, Inc.). The bottom chamber wells receive 27-29 μl of Dulbecco's Modified Eagle Medium (“DMEM”) alone (baseline) or medium containing chemo-attractant (bFGF, VEGF or Swiss 313 cell conditioned medium). The top chambers receive 45 μl of a HUVEC cell suspension (1×10⁶ cells/ml) prepared in DMEM+1% Bovine Serum Albumin (“BSA”) with or without test compound. After 5 hours incubation at 37° C. the membrane is rinsed in Phosphate Buffer Saline (“PBS”), fixed and stained in Diff-Quick solutions. The filter is placed on a glass slide with the migrated cells facing down and cells on top are removed using a Kimwipe. The testing is performed in 4-6 replicates and five fields are counted from each well. Negative unstimulated control values are subtracted from stimulated control and drug treated values and data is plotted as mean migrated cell±Standard Deviation. IC₅₀ is calculated from the plotted data and is reported in Table 8. Graphical results are shown in FIG. 10.

TABLE 8

| | Compound 1 |
|---|---|
| Measurement (HUVEC) | IC₅₀ Concentration (μM) |
| Growth Inhibition | 2.05 |
| Cord Formation | 2.63 |
| Migration | 0.53 |

Having described specific embodiments of the present invention, it will be understood that many modifications thereof will readily appear or may be suggested to those skilled in the art, and it is intended therefore that this invention is limited only by the spirit and scope of the following claims.

1. A method of treating leukemia, carcinoma, melanoma, and/or sarcoma, comprising administering to a mammal a therapeutically effective amount of a compound represented by the following Formula (I) or salt, hydrate, or solvate thereof: 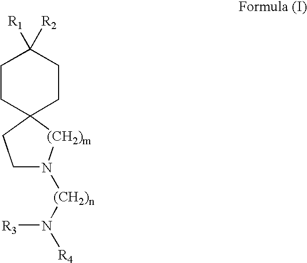
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group.

2. The method of claim 1 wherein at least one of said R₃ or R₄ includes alkyl.

3. The method of claim 1 wherein R₃ and R₄ independently represent a hydrogen atom or a straight chain alkyl having no less than 1 and no more than 3 carbon atoms; or R₃ and R₄ together with the nitrogen form a 5- to 8-member heterocyclic group.

4. The method of any one of the proceeding claims further comprising the administration of a chemotherapeutic or potentiating agent.

5. The method of claim 4, wherein the chemotherapeutic or potentiating agent is selected from triprolidine or its cis-isomer, procodazole, 1H-Benzimidazole carbamate-2-propanoic acid; propazol, monensin, bromodeoxyuridine, dipyridamole, indomethacin, metoclopramide, 7-thia-8-oxoguanosine, N-solanesyl-N,N′-bis(3,4-dimethoxybenzyl)ethylenediamine, leucovorin, heparin, N-[4-[(4-fluorphenyl)sulfonly]phenyl]acetamide, heparin sulfate, cimetidine, vitamin A, 2′-deoxycoformycin, or dimethyl sulfoxide.

6. The method of any one of the proceeding claims wherein the compound is N,N-diethyl-8,8-dipropyl-2-azaspiro[4,5]decane-2-propanamine; or a pharmaceutically acceptable salt, hydrate or solvate thereof.

7. The method of any one of the proceeding claims wherein the compound is administered orally.

8. The method of any one of the proceeding claims wherein the compound is administered parenterally.

9. The method of any one of the proceeding claims wherein from about 0.05 to about 100 mg/kilogram of total body weight of the compound are administered per day.

10. The method of any one of the proceeding claims wherein said mammal is a human.

11. A method of treating cancer comprising administering to a mammal a therapeutically effective amount of a N,N-diethyl-8,8-dipropyl-2-azaspiro[4,5] decane-2-propanamine dimaleate.

12. A method of treating cancer comprising administering to a mammal a therapeutically effective amount of a compound represented by the following Formula (I) or salt, hydrate, or solvate thereof: 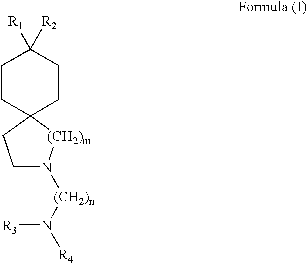
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group;
wherein said cancer includes Hodgkin's Disease, Non-Hodgkin's Lymphoma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

13. A method of suppressing or retarding angiogenesis in a cancer or a tumor, comprising administering to a mammal a therapeutically effective amount of a compound represented by the following Formula (I) or salt, hydrate, or solvate thereof: 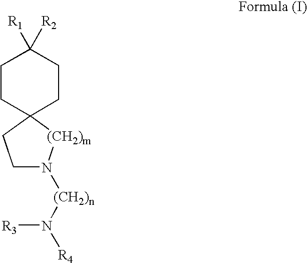
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group.

14. A method for accelerating the rate of apoptosis in cancer cells comprising treating said cells with a therapeutically effective amount of a compound represented by the following Formula (I) or salt, hydrate, or solvate thereof: 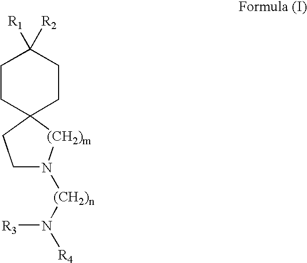
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group.

15. A method of inhibiting the proliferation of cancer cells comprising treating said cells with a therapeutically effective amount of a compound represented by the following Formula (I) or salt, hydrate, or solvate thereof: 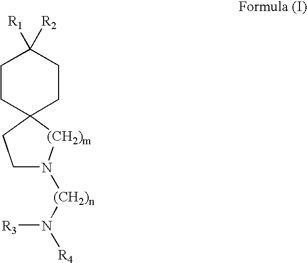
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group.

16. A method of decreasing the secretion of VEGF in cancer cells comprising treating said cells with a therapeutically effective amount of a compound represented by the following Formula (I) or salt, hydrate, or solvate thereof: 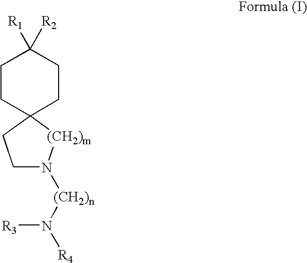
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group.

17. A kit for treating cancer comprising administering to a mammal a therapeutically effective amount of a compound represented by the following Formula (I) or salt, hydrate, or solvate thereof: 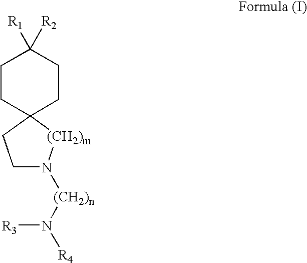
wherein:
n represents a number from 3 to 7;
m represents a number from 1 to 2;
R₁ and R₂ independently represent a hydrogen atom or are a substituted or unsubstituted, branched or unbranched or cyclic, alkyl provided that the total number of carbon atoms represented by R₁ and R₂ when taken together is no less than 5; or
R₁ and R₂ together independently represent a cyclic alkyl group having no less than 3 or no more than 7 carbon atoms;
R₃ and R₄ independently represent a hydrogen atom or a saturated or unsaturated, substituted or unsubstituted, branched or unbranched or cyclic, hydrocarbyl radical, or R₃ and R₄ together with the nitrogen represent at least a 4-member heterocyclic group; and instructions on a dosage regimen.

18. The kit of claim 17 wherein the compound is provided in discrete quantities.

19. The kit of claim 17 wherein the kit is designed for administration to humans.

20. The kit of claim 17 wherein the instruction provide notations specific to certain types of cancer.
